(19) United States
(12) Patent Application Publication
(10) Pub. No.: US 2006/0136266 A1
(43) Pub. Date: Jun. 22, 2006

(54) Medicinal product order processing system

(75) Inventors: Lionel Tarassenko, Oxford (GB); Neil William Townsend, Oxford (GB); Philip Stephen Jones, Aylesbury (GB)

Correspondence Address: NIXON & VANDERHYE, PC, 901 NORTH GLEBE ROAD, 11TH FLOOR, ARLINGTON, VA, 22203, US

(73) Assignee: e-SAN Limited, Oxford (GB)

(21) Appl. No.: 11/015,735

(22) Filed: Dec. 20, 2004

(51) Int. Cl. G06F 19/00

(52) U.S. Cl. 705/3; 705/28

(57) ABSTRACT

A repeat prescription ordering system for allowing patients requiring resupply of medication or medical products to access a server using a portable communications and data processing device such as a smart phone or personal digital assistant. The server supplies to the patient a list of medication and medical products which they are authorized to order. The patient can select the products required, and the order is logged by the server and allocated to a supplier for completion of the order. The server maintains an estimate of the amount of medication or medical product held by the patient, this being based on the prescribed dosage regimen and information entered by the patient on their usage and, optionally, on checks on their own health. The patient may be alerted when the estimate indicates that their supplies are running low. The estimate is allowed to go below zero, this implying a possible departure from the prescribed medication regimen.

### BACKGROUND OF THE INVENTION

The present invention relates to a system for processing orders for prescribed medicinal products.

Patients suffering from long-term or chronic medical conditions, such as diabetes, asthma, hypertension, heart disease and many others, typically have to take medication on a regular basis. Such medication is typically prescribed by a qualified medical practitioner, and this may be on a “repeat” basis, such that the patient can obtain a resupply of the medication when his or her supplies are running low by contacting a pharmacy or other supplier of medicinal products without the need for a detailed examination by the medical practitioner again. Such re-supplies are known as repeat prescriptions. For example, a current routine in the UK for obtaining a repeat prescription is that the patient is provided with a paper slip which, when the repeat prescription is required, the patient delivers to the medical practitioner. A few days later the patient returns to the medical practitioner to obtain the prescription which he can take to the pharmacy and obtain the medication. Alternatively, patients sometimes attend the pharmacy directly, who contact the medical practitioner, and then at a later date has the prescribed medicament ready for collection.

However, there are a number of aspects of this process which are inconvenient and which can lead to poor compliance with the prescribed regime which can interfere with successful control of a patients condition.

Firstly, it can be inconvenient for a patient to attend the premises of a supplier of medicinal products not only to place the order for resupply, but also then to collect the medicaments. This inconvenience tends to lead to patients being supplied with large quantities of medicaments so that they will last a significant length of time, to reduce the number of visits that the patient has to make for order and collection of the medicaments. However, patients frequently do not complete their courses of medication, for example because of undesirable side-effects or because of forgetfulness, and in that case a large supply of medicament may be wasted. Further, it can be dangerous for patients to keep large quantities of certain medicinal products.

Further, some patients do not take their medication at the specified dosage. Such patients may be unwilling to disclose this to the medical practitioner, but generally information about the number of repeat prescriptions obtained is not available to the medical practitioner.

Also, there are some medical conditions in which the dose of medication is varied as necessary, rather than at a regular dosage. In such a case it can be difficult for a patient to obtain a resupply of medication when needed, and difficult for medical practitioners to monitor the amount of medication taken.

### SUMMARY OF THE INVENTION

It is an object of this invention to provide a system which facilitates such a repeat prescription process, in particular by making it more convenient for the patient, but additionally by improving the quality of the monitoring of the patients usage of medication.

Accordingly the present invention provides a system for processing orders for prescribed medicinal products comprising:

a portable communications and data processing device for use by a patient for whom one or more medicinal products have been prescribed, the portable communications and data processing device carrying a prescription ordering application; a server comprising data processing means and a database storing information about the patient, the one or more prescribed medicinal products, and which of a plurality of suppliers of prescribed medicinal products is designated to supply said one or more prescribed medicinal products to said patient; and

data processing terminals connected to said server for use by said suppliers of prescribed medicinal products.

Preferably, on initiation of said prescription ordering application, said prescription ordering application controls the portable communications and data processing device to communicate with said server via a communications network to automatically provide a unique identifier to the server,

in response the server supplies to the portable communications and data processing device a list of only the medicinal products prescribed to that patient and the portable communications and data processing device displays that list, and

in response to operation of the portable communications and data processing device by a patient to select items from the displayed list, the prescription ordering application controls the portable communications and data processing device to send an order for the selected items to the server, said server making said list available for display on the terminal of said designated supplier of prescribed medicinal products.

The server preferably maintains in the database an estimate of the current stock of the prescribed medicinal products held by the patient. This estimate may be based on the prescribed starting amount and dosage, but it may also incorporate a record of medications taken entered by the patient into the portable communications and data processing device and, where applicable, a record of health checks entered by the patient into the portable communications and data processing device. For example, some chronic conditions such as diabetes and hypertension involve the patient making a check on their own health at regular intervals (for example blood glucose level or blood pressure). The results of these checks may be entered automatically or manually into the portable communications and data processing device for transmission to and storage at a database in an electronic health monitoring system. In such a system a record of medication taken may be generated automatically or kept by the patient. It is this record which can be used in the estimate of the stock of medication held by the patient.

The system can also keep track of other medical supplies required by the patient, for example consumable diagnostic items, and again the stock of these can be monitored in the same way. Such items can also be ordered by the patient in the same way.

The estimate of stock may be based on a statistical combination of several of the above factors.

Further, the estimate is allowed to be less than zero. The reason for this is that patients may start with a stock of medication or medical products which the system is unaware of. As the patient uses these, and also the prescribed or subsequently supplied products, the patient may appear to use more than they ordered. This also allows the system to estimate how many days the patient has gone without taking medication. This estimate may be reset after confirmation to the system that a new supply has been collected by the patient.

The server can automatically supply to the terminal at the medical supplier a list of patients whose supplies of medicinal product are running low or have been exhausted. Further, a message personal to the patient, listing the product(s) that they are estimated to be short of, or to have used-up, can be set to the patient. The medical practitioners may also be advised of patients of theirs who have exhausted their supply, and seem not to be following the regime prescribed for them. These steps improve the chances of the patients following the prescribed regime without accidentally running out of a medicament, and also allows medical intervention if a patient ceases to follow the regime. The predetermined amount below which such messaging or warning is triggered can be a certain number of days supply remaining, i.e. can be based on the dosage prescribed for the patient.

Optionally the message to the patient warning of low supply can cause the prescription ordering application to initiate automatically on the portable communications and data processing device. This avoids the need for the patient to remember to order new supplies when the supplies are running low.

The current stock of medication held by the patient can be viewed by the supplier and also the medical practitioner.

The terminal at the supplier's end can display to the supplier a list of pending orders for prescribed medicinal products assigned by the server to that supplier. The supplier can approve or reject each order, and can indicate when the orders are ready for collection. Appropriate status messages are sent automatically by the server to the patients indicating to them the progress of their order, and when the medication is ready for collection or delivery. The fact that the supplier can see the stocks held by patients allocated to them, and also can see a list of patients whose stocks are running low allows the supplier to improve their stock control processes.

The invention extends to the portable communications and data processing device carrying the prescription ordering application, and also to the prescription ordering application itself, which may be in the form of a computer program. The portable communications and data processing device may be a smart phone, GPRS phone or personal digital assistant which communicates with the server via a wireless network, such as one of the cellular telephone networks.

### BRIEF DESCRIPTION OF THE DRAWINGS

FIG. 1 is an overall view of the system in accordance with one embodiment of the inventions;

FIG. 2 is a schematic view of a portable communications and data processing device in the system of FIG. 1;

FIG. 3 is a schematic diagram of the server in the system of FIG. 1;

FIG. 4 is a flow diagram of the process between the patient and the server in an embodiment of the invention;

FIG. 5 is a flow diagram of the processing between the pharmacy and server in an embodiment of the invention;

FIG. 6 is a flow diagram of the interaction between the patient and the portable communication and data processing device in an embodiment of the invention;

FIG. 7 is a screen shot of a display on a device in one embodiment of the invention;

FIG. 8 is a screen shot of a display on a device in one embodiment of the invention;

FIG. 9 is a screen shot of a display on a device in one embodiment of the invention;

FIG. 10 is a screen shot of a display screen in a medical supplier's terminal according to an embodiment of the invention;

FIG. 11 is a screen shot of a display screen in a medical supplier's terminal according to an embodiment of the invention;

FIG. 12 is a screen shot of a message in an embodiment of the invention;

FIG. 13 is a screen shot of a message in an embodiment of the invention;

FIG. 14 is a screen shot of another display on a medical supplier's screen according to an embodiment of the invention;

FIG. 15 is another screen shot of another display on a medical supplier's screen according to an embodiment of the invention.

### DETAILED DESCRIPTION

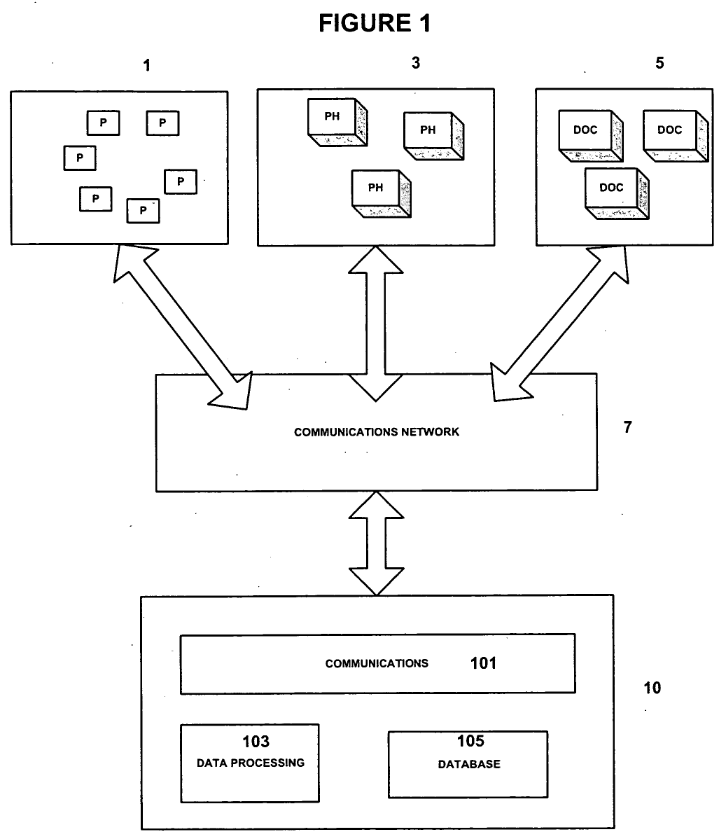

FIG. 1 is a schematic diagram showing the overall system according to one embodiment of the invention. It comprises three groups who interact with the system, the patients 1, the medical product suppliers, such as pharmacies, 3 and medical practitioners 5, such as doctors. In this embodiment each of the patients is supplied with a portable communications and data processing device P, which in this embodiment is a mobile telephone (cellphone) or PDA with a communication capability. Each of the medical product suppliers is supplied with a terminal PH and each of the medical practitioners is supplied with a terminal DOC. The terminals and the portable communications and data processing devices communicate via a communications network 7 with a server 10. The communications network includes cellular phone networks, the internet, conventional telephone networks and so on. The server 10 includes a communications interface 101, a data processor 103 and a database 105.

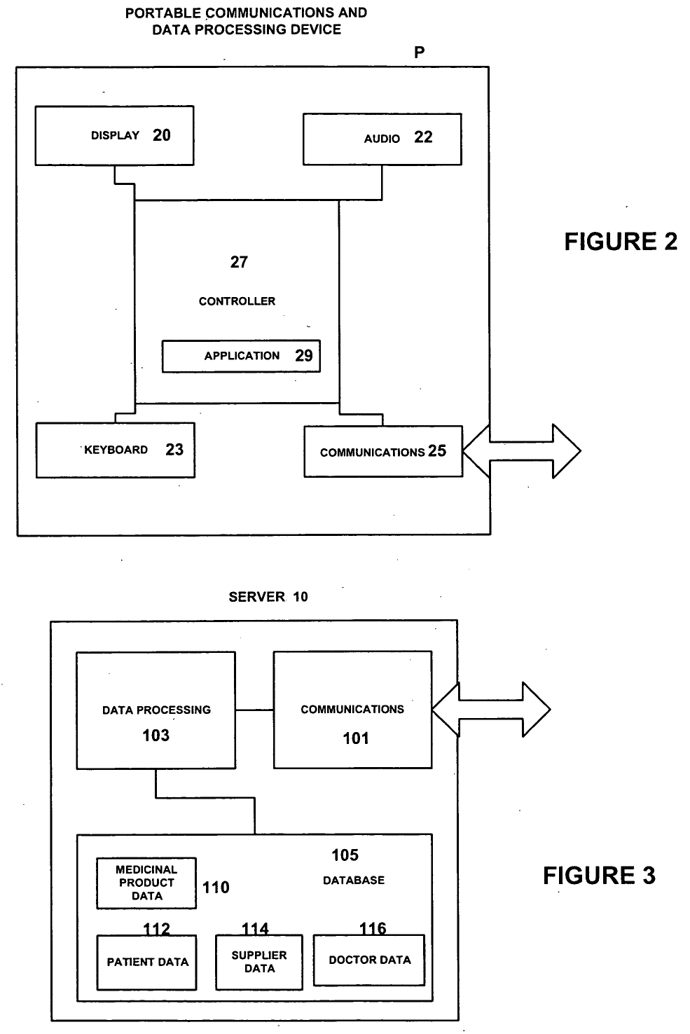

FIG. 2 illustrates the portable communications and data processing device P schematically. This can be a GPRS or smart phone and, as is conventional, includes a display, such as a screen 20, audio input and output 22, a keypad 23 and a communications interface 25. These are under the control of a controller 27. In accordance with this embodiment of the invention the device is provided with a prescriptions ordering application 29, conveniently in the form of a software application run by the controller 27.

FIG. 3 illustrates the server 10 in more detail, and as illustrated there the database 105 stores information necessary for the operation of the system. In particular it stores data 110 on medicinal products, data 112 on the patients who subscribe to the system, data 114 on the medical products suppliers and data 116 on the medical practitioners. The information stored in the patient, supplier and practitioner databases comprise identity and log-on information so that these parties may log-on to the system, have their identity confirmed and then communicate securely with the system. It also stores data on the relationship between the parties, such as which doctors are responsible for which patients and which suppliers will be supplying which orders, as well storing the data on the medical products required by each patient and the current stock held by each patient.

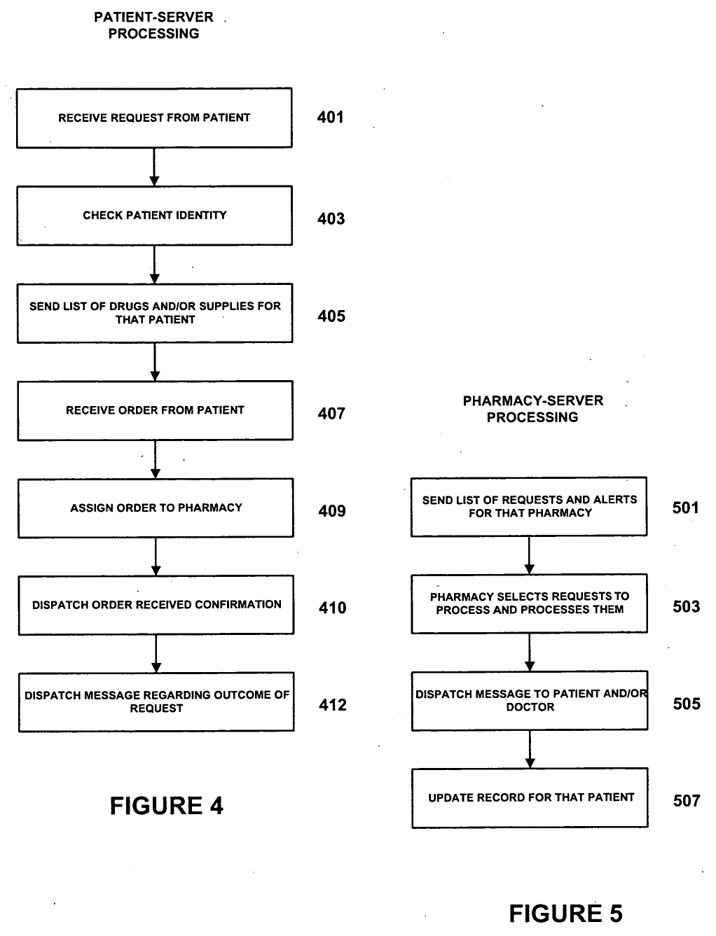

An example of the operation of the system in a typical order for medical supply from a patient will now be explained with reference to FIGS. 4 to 15.

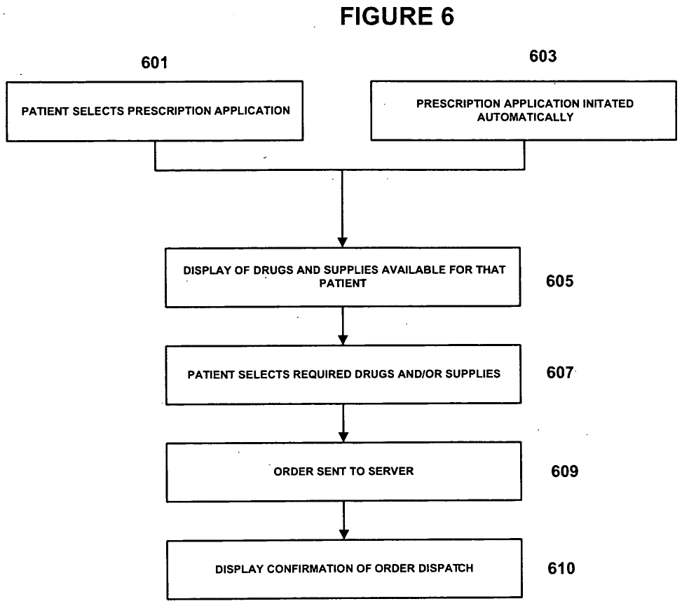

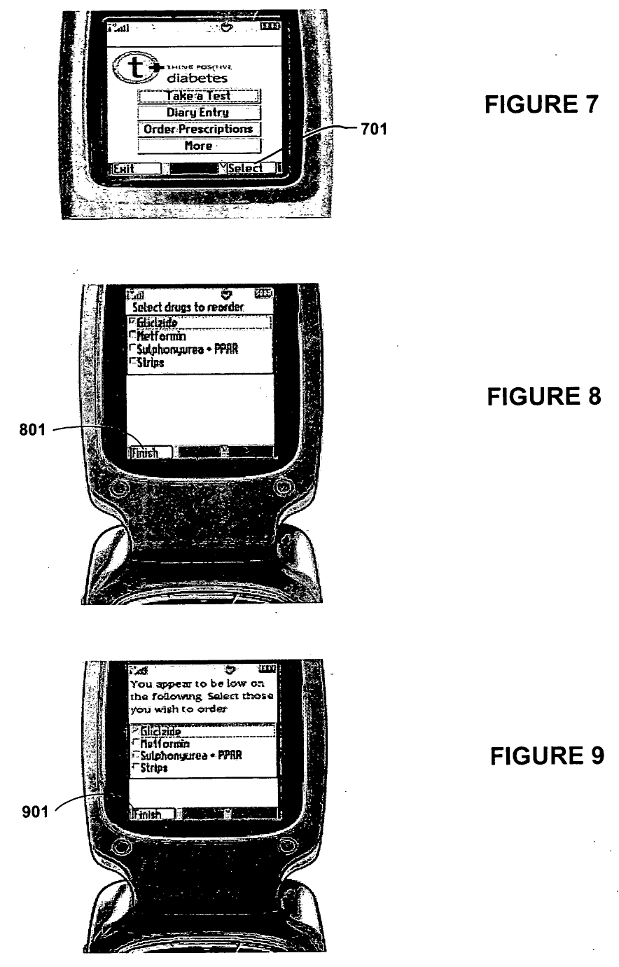

As illustrated in FIG. 6 as a first step the prescription application on the portable communications and data processing device P is either selected by the patient at step 601 or initiated automatically at step 603, for example by a message from the server 10 indicating that the patient's supply of a medical product is running low. FIG. 7 illustrates an initial screen-shot of the display on the device P in which the prescription ordering application is initiated by selection of “order prescriptions” by scrolling in the list and use of the “select” the “button” 701.

The portable communications and data processing device P will then display a list of drugs and supplies available for that particular patient. This list may either be stored on the device P itself, or retrieved from the server 10. In the case of retrieval from the server 10, as this constitutes the first contact with the server, the process includes an automatic, and invisible to the patient, check 403 on the patient identity following the reception at step 401 of the request from the patient. This check consists, for example, of an identification code and an authentication code. The list of drugs and/or supplies available for that patient is then sent by the server to the device P.

As indicated in step 605 the list of drugs and supplies available for that patient are then displayed on the device P and a typical display is shown in FIG. 8. The display illustrated in FIG. 8 includes both prescription for drugs and also other medical products such as consumable items used by the patient in monitoring or managing their condition. For example, diabetes suffers may have to check their blood glucose levels regularly, which typically requires use of a disposable absorbent strip and lancet each time.

FIG. 9 illustrates the alternative form of display which occurs in the case of automatic initiation of the prescription ordering application. In this case the patient is warned that they appear to be low on certain of the prescription drugs and/or medical supplies. This list indicates just those products for which the quantity is estimated to be low, but may alternatively may include the complete list available to the patient.

As indicated in step 607 the patient then selects the products that they wish to order, e.g. by ticking the appropriate boxes in the lists indicated in FIG. 8 or 9. Then by use of the “finish” button 801, 901 the order is sent to the server 10 step 609.

On reception at step 407 of the order from the patient, the server assigns in step 409 the order to a particular medical supplier by reference to its stored data on patients and suppliers. It also dispatches an order received confirmation to the device P, which is displayed at step 610.

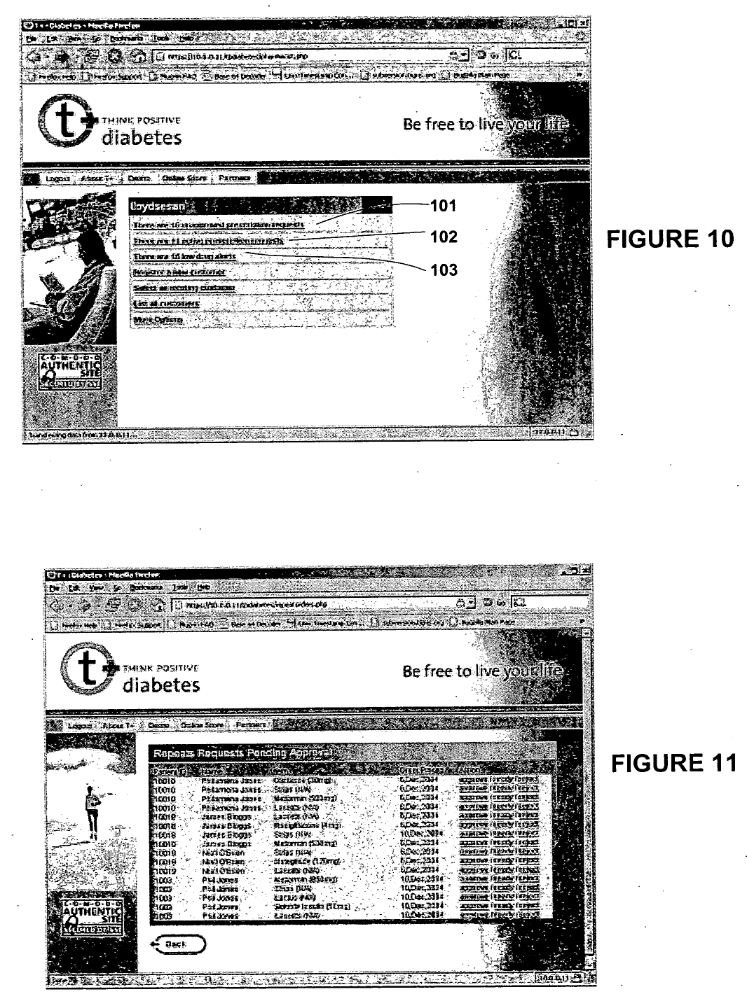

The suppliers 3 in FIG. 1 log-on to the system regularly, e.g. for the duration of business hours, and subject to the usual log-on and identity checks the server sends at step 501 a list of the requests and alerts it has allocated to that particular supplier. FIG. 10 illustrates the basic display to the supplier and it can be seen that it indicates the number of unapproved prescription orders 101, that is to say orders which have not yet been processed, the number of active prescription orders 102, that is to say orders which have been approved or are ready for collection but have not yet been collected, and the number of low drug alerts 103. The low drug alerts are an indication of those patients whose supplies as estimated by the server 10 are running low.

As illustrated in FIG. 10 the supplier is also able to register new patients (or customers), access the records of an existing patient and list all of their current patients.

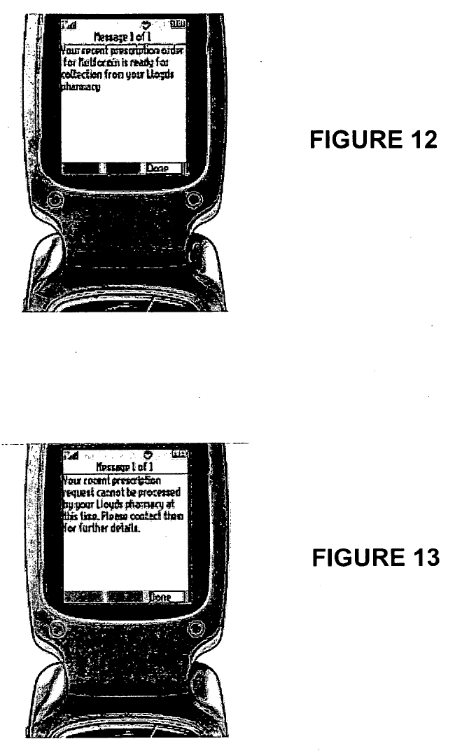

FIG. 11 illustrates the display for the currently unapproved prescription requests 101. It can be seen that it lists the patient's name, the item required, the date of placement of the order and the patient ID number, and gives the pharmacist options for approval of the order, indication that an already approved order is ready for collection, or rejection of the order. The pharmacist therefore selects each order and processes it as illustrated in step 503 on FIG. 5. An appropriate message is sent to the patient and/or medical practitioner at step 505 via the server and the communications network. Thus as illustrated in FIG. 12 the message may indicate that the prescription order is ready for collection, or in FIG. 13 may indicate that the prescription order has been refused and invite the patient to contact the supplier or the medical practitioner. In the case of refusal, a suitable message may also be sent to the medical practitioner registered for that patient by reference to the stored information in the server's database.

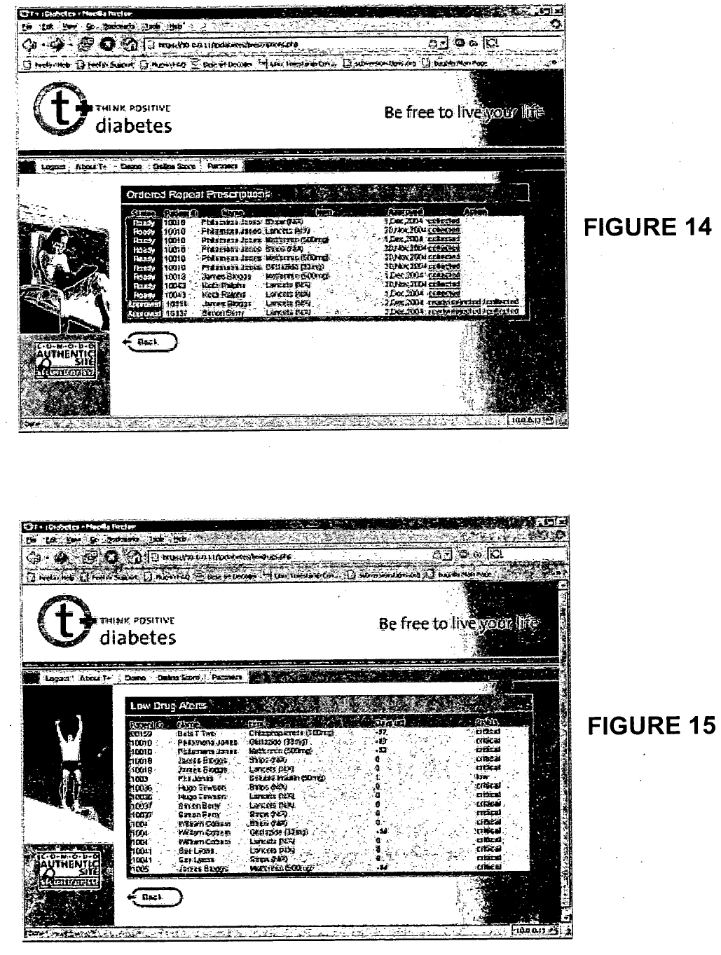

When the order is collected (or delivered), this is also registered at the supplier as indicated in FIG. 14 and the patient record is updated on the server as indicated at step 507.

FIG. 15 illustrates the display at the supplier of the low supplies alerts 103. It can be seen that depending on the estimated level the indication can be merely low (implying that the patient still has some product available), or “critical” implying that, as far as the system is concerned, the patient has exhausted their supply.

This estimate of the current stock of product held by the patient is maintained by the server 10 using the database 105 and data processor 103. As illustrated in FIG. 15 the estimate is displayed in terms of the number of “days left”, that is to say the number of days before the supplies are exhausted. The estimate is maintained by the application of certain rules to a medication count. The count is initialized by the amount of medication supplied to the customer on first registration with the system. It is triggered by the delivery or collection of the product by the customer (thus avoiding pointless processing for customers who do not collect their medication). The server stores information on the products and also on the prescription, and thus can deduct from the initial amount the amount of product which the customer should have used according to the prescribed regimen. Further, the patient can be asked to input into the device P the amount of medication they have taken. This information can either be direct in the form of asking the customer to input exactly how much of the particular product they have used, or implicit based on the normal consumption of product for a health check whose results are entered by the customer into the system. For example, in the case of a patient suffering from diabetes, making the checks mentioned above using lancets and disposable strips, the entry by the patient into an electronic health monitoring system of three sets of results implies the use of three strips and lancets. Thus integration of the prescription ordering system of the present invention with the applicants health monitoring system, for example as disclosed in co-pending patent application number PCT/GB2003/004029, means that this implied consumption information is available at the server 10. By the use of this information as well, the estimate of current stock can be improved.

It is important to note that the estimate is allowed to count down below zero. This is illustrated in FIG. 15. The reason for this is that customers may have supplies of medicament or medical product which are “unknown” to the system. This negative counting may be reset when the customer reorders on the basis that such reordering provides a clear zero start point for the system. When a customer collects their prescription, their known supply is reset to either the quantity they collect, or the sum of that quantity plus the current estimate of stock, whichever is greater.

It will be appreciated from the above that the system does not apply only to the ordering of prescribed medication, but also to medical products used by the customer in the course of the management of their condition, and to non-prescription medicaments. Furthermore, prescribed medication may be taken on a regular or irregular basis depending on whether it is required all the time or only as necessary according to the symptoms felt by the patient.

In the present invention the messages to the patient are sent by a bespoke messaging system designed to work in the present embodiment within a GPRS application. However they may be sent by the short messaging service (SMS) or e-mail or other means if desired.

The level at which the automatic initiation of the prescription ordering system occurs may be set as desired. This may depend on the severity of the patients condition, but in the illustrated embodiment is set at seven days supply remaining.

In the above description it is envisaged that the supplier does not need to contact the medical practitioner for confirmation of the repeat order. However, should such confirmation be required this can either occur automatically by the patient request first being alerted to the medical practitioner DOC for approval, and then being passed to the supplier PH for processing, or the supplier PH may be provided with an indication on the display shown in FIG. 11 that authorization needs to be sought from the medical practitioner. Because the server stores data on which medical practitioners handle which patients, the request for confirmation can be supplied simply by the server to the appropriate practitioner either automatically or when selected to do so by the supplier.

Advantages of this system are that the patient can easily monitor the estimates of the medication they hold by accessing that information either stored on the device P or by accessing it on the server 10 using the device P, and can conveniently order more supplies when required. Further, because the system maintains an estimate of the remaining supply, the patient is less likely to run out of product. Further, the medical practitioner and supplier can monitor the consumption of medication and supplies by the patients and thus be alerted to those who are running low or have apparently run out of supplies. Running out of supplies is an indicator either that the patient is not sticking to the medication regimen (which needs attention by the medical practitioner) or that they are finding alternative sources of medication (which again would require investigation by the medical practitioner).

The interaction with the system of the medical practitioner allows straightforward changes to be made in the medication regimen without the need to see the patient. Thus a slight increase/decrease in medication, or change in frequency, could be agreed between the practitioner and the patient (e.g. by telephone) in which the case this can be entered by the practitioner, stored on the server and reflected in the ordering process available for that patient. In some jurisdictions only the medical practitioner may be permitted to do so, but the supplier can also be given the authorisation to do this, either to the same or to a more limited extent.

It should be noted that communications between the parties to the system are secured by means of requiring log-on identity checks and encrypted transmission as is conventional. Further, it should be noted that the interactions of the parties with the system are stored, rather than being discarded, thus resulting in the creation of an audit trail for each order.

The terminals useable by the suppliers 3 and practitioners 5 may be conventional personal computers, or portable communications and data processing devices such as smart phone, GPRS phones or PDAs. These run a prescription ordering application in a similar way to the patient devices P but which provides the functionality required by them. The server 10 may take the form of a personal computer, again running a prescription ordering application to provide the data processing and data storing functionality required by the system.

We claim:

1. A system for processing orders for prescribed medicinal products comprising:
a portable communications and data processing device for use by a patient for whom one or more medicinal products have been prescribed, the portable communications and data processing device carrying a prescription ordering application; a server comprising data processing means and a database storing information about the patient, the one or more prescribed medicinal products, and which of a plurality of suppliers of prescribed medicinal products is designated to supply said one or more prescribed medicinal products to said patient; and
data processing terminals connected to said server for use by said suppliers of prescribed medicinal products.

2. A system according to claim 1 wherein on initiation of said prescription ordering application, said prescription ordering application controls the portable communications and data processing device to communicate with said server via a communications network to automatically provide a unique identifier to the server,
in response the server supplies to the portable communications and data processing device a list of only the medicinal products prescribed to that patient and the portable communications and data processing device displays that list, and
in response to operation of the portable communications and data processing device by a patient to select items from the displayed list, the prescription ordering application controls the portable communications and data processing device to send an order for the selected items to the server, said server making said list available for display on the terminal of said designated supplier of prescribed medicinal products.

3. A system according to claim 1 wherein the server maintains in the database an estimate of the current stock of the prescribed medicinal products held by the patient.

4. A system according to claim 3 wherein said estimate is based on the prescribed starting amount and dosage of said one or more medicinal products.

5. A system according to claim 3 wherein said estimate is based on a record of usage entered by said patient into said portable communications and data processing device.

6. A system according to claim 3 wherein said estimate is based on a record of health checks entered by said patient into said portable communications and data processing device.

7. A system according to claim 3 wherein said estimate is based on a statistical combination of two or more of: the prescribed starting amount and dosage, the patient-entered record of usage, and the patient-entered record of health checks.

8. A system according to claim 3 wherein the estimate can be less than zero.

9. A system according to claim 8 wherein a less than zero estimate is reset after confirmation to said server from said terminal of supply to said patient of said one or more prescribed medicinal products.

10. A system according to claim 3 wherein said server automatically supplies to said terminal a list of patients whose estimated current stock of the prescribed medicinal products is less than a predetermined amount.

11. A system according to claim 10 wherein said predetermined amount is based on the prescribed dosage.

12. A system according to claim 3 wherein said server automatically supplies to a medical practitioner information on the identity of patients whose estimated current stock of the prescribed medicinal products is less than a predetermined amount.

13. A system according to claim 3 wherein said server automatically supplies a message to the portable communications and data processing device of a patient whose estimated current stock of the prescribed medicinal products is less than a predetermined amount.

14. A system according to claim 13 wherein said message causes automatic initiation at portable communications and data processing device of said prescription ordering application.

15. A system according to claim 3 wherein said estimate is displayable on said portable communications and data processing device and on said terminal.

16. A system according to claim 1 wherein the server supplies to said terminal a list of pending orders for prescribed medicinal products designated to be supplied by that supplier

17. A system according to claim 16 wherein the terminal includes selection means for allowing each order to be approved or rejected, in response to which a corresponding status message is automatically sent from said server to said portable communications and data processing device.

18. A system according to claim 16 wherein the selection means further allows an order to be indicated as ready for collection, in response to which a status message is automatically sent from said server to said portable communications and data processing device.

19. A portable communications and data processing device carrying a prescription ordering application, for use in the system for processing orders for prescribed medicinal products as defined in claim 1.

20. A prescription ordering application for a portable communications and data processing device for use in the system for processing orders for prescribed medicinal products as defined in claim 1.
